(19) United States
(12) Patent Application Publication
(10) Pub. No.: US 2026/0355170 A1
(43) Pub. Date: Nov. 12, 2026

(54) Treatment of Endometrial Cancer with MDM2 Inhibitors

(71) Applicant: Kartos Therapeutics, Inc., Redwood City, CA (US)

(72) Inventor: Wayne Rothbaum, Delray Beach, FL (US)

(73) Assignee: Kartos Therapeutics, Inc., Redwood City, CA (US)

(21) Appl. No.: 19/489,748

(22) PCT Filed: Jun. 5, 2024
(86) PCT No.: PCT/US2024/032561

§ 371 Date: Dec. 3, 2025

Related U.S. Application Data
(60) Provisional application No. 63/471,134, filed on Jun. 5, 2023.

(51) Int. Cl. A61K 31/451; A61P 35/00

(52) U.S. Cl. CPC A61K 31/451; A61P 35/00

(57) ABSTRACT

Therapeutic methods and pharmaceutical compositions for treating endometrial cancer in a human are described. The methods comprise the step of administering to a human in need thereof a therapeutically effective amount of a MDM2 inhibitor. The pharmaceutical compositions comprise a therapeutically effective amount of a MDM2 inhibitor.

### FIELD OF THE DISCLOSURE

Methods of treating endometrial cancer in a human using a Mouse double minute 2 homolog (MDM2) inhibitor are disclosed herein.

### BACKGROUND

p53 is a tumor suppressor and transcription factor that responds to cellular stress by activating the transcription of numerous genes involved in cell cycle arrest, apoptosis, senescence, and DNA repair. Unlike normal cells, which have infrequent cause for p53 activation, tumor cells are under constant cellular stress from various insults including hypoxia and oncogene activation. As p53 would normally stop cell proliferation and cull abnormal cells to suppress oncogenesis and tumor growth, there is a strong selective advantage for inactivation of the p53 pathway in tumors, and it has been proposed that suppressing p53 function may be a prerequisite for tumor survival. In support of this notion, three groups of investigators have used mouse models to demonstrate that absence of p53 function is a continuous requirement for the maintenance of established tumors (Cheok, Seeking synergy in p53 transcriptional activation for cancer therapy. Discov Med., 2012, 14(77), 263-271; Qian, Tumor suppression by p53: making cells senescent. Histol Histopathol. 2010, 25(4), 515-26; Beraza, Restoration of p53 function: a new therapeutic strategy to induce tumor regression? Hepatology, 2007, 45(6), 1578-9). When the investigators restored p53 function to tumors with inactivated p53, the tumors regressed. [0003]p53 is inactivated by mutation and/or loss in 50% of solid tumors and 10% of liquid tumors. Other key members of the p53 pathway are also genetically or epigenetically altered in cancer. MDM2, an oncoprotein, inhibits p53 function, and it is activated by gene amplification at incidence rates that are reported to be as high as 10%. MDM2, in turn, is inhibited by another tumor suppressor, p14ARF. It has been suggested that alterations downstream of p53 may be responsible for at least partially inactivating the p53 pathway in p53^{WT} tumors (p53 wild type). In support of this concept, some p53^{WT} tumors appear to exhibit reduced apoptotic capacity, although their capacity to undergo cell cycle arrest remains intact. One cancer treatment strategy involves the use of small molecules that bind MDM2 and neutralize its interaction with p53. MDM2 inhibits p53 activity by three mechanisms: 1) acting as an E3 ubiquitin ligase to promote p53 degradation; 2) binding to and blocking the p53 transcriptional activation domain; and 3) exporting p53 from the nucleus to the cytoplasm. All three of these mechanisms would be blocked by neutralizing the MDM2-p53 interaction. In particular, this therapeutic strategy could be applied to tumors that are p53^{WT}, and studies with small molecule MDM2 inhibitors have yielded promising reductions in tumor growth both in vitro and in vivo. Further, in patients with p53-inactivated tumors, stabilization of wild type p53 in normal tissues by MDM2 inhibition might allow selective protection of normal tissues from mitotic poisons. As used herein, MDM2 means a human MDM2 protein and p53 means a human p53 protein. It is noted that human MDM2 can also be referred to as HDM2 or hMDM2. Several MDM2 inhibitors are in human clinical trials for the treatment of various cancers.

Endometrial cancer is a cancer that starts in the layer of cells that form the lining (endometrium) of the uterus. Treatment approaches include surgery, chemotherapy, radiation therapy, or a combination of these approaches. Advanced and/or recurrent endometrial cancer is frequently treated with surgery followed by chemotherapy, radiation therapy, or both. While early-stage disease is associated with good prognosis, treatment approaches for advanced and/or recurrent endometrial cancer suffer from toxicity and limited efficacy. Thus, there is a need for effective therapies to improve median survival and overall outcome.

The present disclosure relates to methods of treating endometrial cancer in a human with an MDM2 inhibitor.

### SUMMARY

The present disclosure relates to methods of treating endometrial cancer in a human. The methods comprises administering to the human a therapeutically effective amount of a MDM2 inhibitor. In one aspect, the present disclosure relates to a method of treating endometrial cancer comprising the step of administering to a human in need thereof a therapeutically effective amount of a MDM2 inhibitor, wherein the human is previously treated with chemotherapy and obtains a clinical benefit, wherein administration of the MDM2 inhibitor maintains the clinical benefit. In an embodiment, the MDM2 inhibitor is a compound of Formula (I):

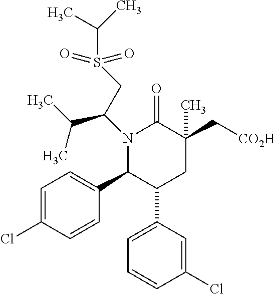 [SMILES: CC(C)[C@@H](CS(=O)(=O)C(C)C)N1C(=O)[C@@](C)(CC(=O)O)C[C@H](c2cccc(Cl)c2)[C@H]1c1ccc(Cl)cc1]

or a pharmaceutically acceptable salt thereof.

In an embodiment, the clinical benefit is selected from the group consisting of objective response (OR), overall survival (OS), progression free survival (PFS), complete response (CR), partial response (PR), stable disease (SD), and combinations thereof. In an embodiment, the clinical benefit is CR, PR, or SD. In an embodiment, the clinical benefit is CR or PR.

In an embodiment, the cancer is responsive to chemotherapy.

In an embodiment, the cancer has not progressed after chemotherapy. In an embodiment, the cancer has not progressed after treatment with a PD-1 inhibitor or a PD-L1 inhibitor. In an embodiment, the cancer has not progressed after chemotherapy and treatment with a PD-1 inhibitor or a PD-L1 inhibitor.

In an embodiment, the human received an induction treatment with chemotherapy. In an embodiment, the human received an induction treatment with immunotherapy. In an embodiment, the human received an induction treatment with a combination of chemotherapy and immunotherapy. In an embodiment, the immunotherapy in the induction treatment comprises treatment with a PD-1 inhibitor. In an embodiment, the immunotherapy in the induction treatment comprises treatment with a PD-L1 inhibitor. In an embodiment, the human achieved a clinical benefit from the induction treatment.

In an embodiment, the methods further comprise administration of an immunotherapy in combination with the MDM2 inhibitor. In an embodiment, the immunotherapy is a PD-1 inhibitor or a PD-L1 inhibitor.

In an embodiment, the human has advanced or recurrent endometrial cancer. In an embodiment, the human has TP53 wild-type endometrial cancer. In an embodiment, the human has cyclin-dependent kinase inhibitor 2A (CDKN2A)-deleted or inactivated endometrial cancer. In an embodiment, the human has endometrial cancer characterized by CDKN2A dysfunction. In an embodiment, the human has endometrial cancer characterized by one or more CDKN2A mutations. In an embodiment, the human has endometrial cancer characterized by loss of CDKN2A. In an embodiment, the human has endometrial cancer characterized by p16|NK4a promoter hypermethylation and/or p14ARF promoter hypermethylation.

In an embodiment, the MDM2 inhibitor is a pharmaceutically acceptable salt of a compound of Formula (I).

In an embodiment, the compound of Formula (I) is administered once daily at a dose selected from the group consisting of about 15 mg, about 25 mg, about 30 mg, about 50 mg, about 60 mg, about 75 mg, about 90 mg, about 100 mg, about 120 mg, about 150 mg, about 175 mg, about 180 mg, about 200 mg, about 225 mg, about 240 mg, about 250 mg, about 275 mg, about 300 mg, about 325 mg, about 350 mg, about 360 mg, about 375 mg, and about 480 mg. In an embodiment, the compound of Formula (I) is administered twice daily at a dose selected from the group consisting of about 15 mg, about 25 mg, about 30 mg, about 50 mg, about 60 mg, about 75 mg, about 90 mg, about 100 mg, about 120 mg, about 150 mg, about 175 mg, about 180 mg, about 200 mg, about 225 mg, about 240 mg, about 250 mg, about 275 mg, about 300 mg, about 325 mg, about 350 mg, about 360 mg, about 375 mg, and about 480 mg.

In an embodiment, the human is treated with the compound of Formula (I) for a period selected from the group consisting of about 7 days, about 14 days, about 21 days, about 28 days, about 35 days, about 42 days, about 49 days, and about 56 days.

In an embodiment, the compound of Formula (I) is orally administered.

In an embodiment, the therapeutically effective amount of the MDM2 inhibitor is about 180 mg. In an embodiment, the therapeutically effective amount of the MDM2 inhibitor is about 240 mg.

In an embodiment, the human is previously treated with platinum-based therapy. In an embodiment, the human is previously treated with radiation therapy.

In an embodiment, the MDM2 inhibitor is administered intermittently. In an embodiment, the MDM2 inhibitor is administered on days 1-7, followed by discontinuance of administration for days 8-28 on a 28-day cycle, optionally repeating the cycle of administration and discontinuation of administration for 2, 3, 4, 5, 6, 7, 8, 9, or 10 times.

### BRIEF DESCRIPTION OF THE DRAWINGS

The foregoing summary, as well as the following detailed description of the disclosure, will be better understood when read in conjunction with the appended drawings.

FIG. 1A illustrates growth of HEC-151 TP53 WT endometrial cancer cells following MDM2 inhibitor treatment. FIG. 1B illustrates growth of HEC-265 TP53 WT endometrial cancer cells following MDM2 inhibitor treatment. FIG. 1C illustrates growth of HEC-108 TP53 WT endometrial cancer cells following MDM2 inhibitor treatment. DMSO, dimethyl sulfoxide; EC₅₀, half maximal effective concentration; EC₉₀, 90% maximal effective concentration; Cₘₐₓ, maximum serum concentration; μM, micromolar.

### DETAILED DESCRIPTION

While preferred embodiments of the disclosure are shown and described herein, such embodiments are provided by way of example only and are not intended to otherwise limit the scope of the disclosure. Various alternatives to the described embodiments of the disclosure may be employed in practicing the disclosure.

Unless defined otherwise, all technical and scientific terms used herein have the same meaning as is commonly understood by one of skill in the art to which this disclosure belongs.

The terms “administered in combination with” and “co-administration” as used herein, encompass administration of two or more active pharmaceutical ingredients to a subject so that both agents and/or their metabolites are present in the subject at the same time. Co-administration includes simultaneous administration in separate compositions, administration at different times in separate compositions, or administration in a composition in which two or more agents are present.

The term “combination” or “pharmaceutical combination” is defined herein to refer to either a fixed combination in one dosage unit form, a non-fixed combination or a kit of parts for the combined administration where the therapeutic agents may be administered together, independently at the same time or separately within time intervals, which preferably allows that the combination partners show a cooperative, e.g. synergistic effect. Thus, the single compounds of the pharmaceutical combination of the present disclosure could be administered simultaneously or sequentially.

Furthermore, the pharmaceutical combination of the present disclosure may be in the form of a fixed combination or in the form of a non-fixed combination.

The term “effective amount” or “therapeutically effective amount” refers to that amount of an active pharmaceutical ingredient or combination of active pharmaceutical ingredients as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, and other factors which can readily be determined by one of ordinary skill in the art. The term also applies to a dose that will induce a particular response in target cells, (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

The terms “enantiomerically enriched,” “enantiomerically pure,” and “non-racemic,” as used herein, refer to compositions in which the percent by weight of one enantiomer is greater than the amount of that one enantiomer in a control mixture of the racemic composition (e.g., greater than 1:1 by weight). For example, an enantiomerically enriched preparation of the (S)-enantiomer, means a preparation of the compound having greater than 50% by weight of the (S)-enantiomer relative to the (R)-enantiomer, such as at least 75% by weight, such as at least 80% by weight. In some embodiments, the enrichment can be significantly greater than 80% by weight, providing a “substantially enantiomerically enriched,” “substantially enantiomerically pure,” or a “substantially non-racemic” preparation, which refers to preparations of compositions which have at least 85% by weight of one enantiomer relative to the other enantiomer, such as at least 90% by weight, and such as at least 95% by weight. The terms “diastereomerically enriched” and “diastereomerically pure,” as used herein, refer to compositions in which the percent by weight of one diastereomer is greater than the amount of that one diastereomer in a control mixture of diastereomers. In some embodiments, the enrichment can be significantly greater than 80% by weight, providing a “substantially diastereomerically enriched” or “substantially diastereomerically pure” preparation, which refers to preparations of compositions which have at least 85% by weight of one diastereomer relative to other diastereomers, such as at least 90% by weight, and such as at least 95% by weight.

In some embodiments, the enantiomerically enriched composition has a higher potency with respect to therapeutic utility per unit mass than does the racemic mixture of that composition. Enantiomers can be isolated from mixtures by methods known to those skilled in the art, including chiral high pressure liquid chromatography (HPLC) and the formation and crystallization of chiral salts; or some enantiomers can be prepared by asymmetric syntheses. See, for example, Jacques, Enantiomers, Racemates and Resolutions, Wiley Interscience, New York (1981); E. L. Eliel and S. H. Wilen, Stereochemistry of Organic Compounds, Wiley-Interscience, New York (1994).

“Enantiomeric purity” as used herein refers to the relative amounts, expressed as a percentage, of the presence of a specific enantiomer relative to the other enantiomer. For example, if a compound, which may potentially have an (R)- or an (S)-isomeric configuration, is present as a racemic mixture, the enantiomeric purity is about 50% with respect to either the (R)- or (S)-isomer. If that compound has one isomeric form predominant over the other, for example, 80% (S)- and 20% (R)-, the enantiomeric purity of the compound with respect to the (S)-isomeric form is 80%. The enantiomeric purity of a compound can be determined in a number of ways known in the art, including but not limited to chromatography using a chiral support, polarimetric measurement of the rotation of polarized light, nuclear magnetic resonance spectroscopy using chiral shift reagents which include but are not limited to lanthanide containing chiral complexes or the Pirkle alcohol, or derivatization of a compounds using a chiral compound such as Mosher's acid followed by chromatography or nuclear magnetic resonance spectroscopy.

The term “fixed combination” means that the therapeutic agents, e.g., the single compounds of the combination, are in the form of a single entity or dosage form.

The term “IC₅₀” refers to the half maximal inhibitory concentration, i.e. inhibition of 50% of the desired activity. The term “EC₅₀” refers to the drug concentration at which one-half the maximum response is achieved.

“Isomers” are different compounds that have the same molecular formula. “Stereoisomers” are isomers that differ only in the way the atoms are arranged in space—i.e., having a different stereochemical configuration. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon can be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R)- or (S)-. The present chemical entities, pharmaceutical compositions and methods are meant to include all such possible isomers, including racemic mixtures, optically pure forms and intermediate mixtures. Optically active (R)- and (S)-isomers can be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

The term “fixed combination” means that the therapeutic agents, e.g., the single compounds of the combination, are in the form of a single entity or dosage form.

The term “non-fixed combination” means that the therapeutic agents, e.g., the single compounds of the combination, are administered to a patient as separate entities or dosage forms either simultaneously or sequentially with no specific time limits, wherein preferably such administration provides therapeutically effective levels of the two therapeutic agents in the body of the subject, e.g., a mammal or human in need thereof.

“Pharmaceutically acceptable carrier” or “pharmaceutically acceptable excipient” is intended to include any and all solvents, dispersion media, coatings, antibacterial and antifungal agents, isotonic, and absorption delaying agents. The use of such media and agents for active pharmaceutical ingredients is well known in the art. Except insofar as any conventional media or agent is incompatible with the active pharmaceutical ingredient, its use in the therapeutic compositions of the disclosure is contemplated. Supplementary active ingredients can also be incorporated into the described compositions. Unless otherwise specified, or clearly indicated by the text, reference to therapeutic agents useful in the pharmaceutical combination of the present disclosure includes both the free base of the compounds, and all pharmaceutically acceptable salts of the compounds.

The term “pharmaceutically acceptable salt” refers to salts derived from a variety of organic and inorganic counter ions known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid and phosphoric acid. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In selected embodiments, the pharmaceutically acceptable base addition salt is chosen from ammonium, potassium, sodium, calcium, and magnesium salts. The term “cocrystal” refers to a molecular complex derived from a number of cocrystal formers known in the art. Unlike a salt, a cocrystal typically does not involve proton transfer between the cocrystal and the drug, and instead involves intermolecular interactions, such as hydrogen bonding, aromatic ring stacking, or dispersive forces, between the cocrystal former and the drug in the crystal structure.

The terms “QD,” “qd,” or “q.d.” means quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quater in die, four times a day, or four times daily.

“Solvate” refers to a compound in physical association with one or more molecules of a pharmaceutically acceptable solvent.

A “therapeutic effect” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, inhibition of metastases, or any combination thereof.

The term “chemotherapy” refers to a type of cancer treatment that uses one or more anti-cancer drugs (chemotherapeutic agents) as part of a standardized chemotherapy regimen. Chemotherapy may be given with a curative intent (which almost always involves combinations of drugs), or it may aim to prolong life or to reduce symptoms (palliative chemotherapy). Drugs for chemotherapy includes but is not limited to cyclophosphamide, methotrexate, 5-fluorouracil, vinorelbine, doxorubicin, docetaxel, bleomycin, vinblastine, dacarbazine, mustine, vincristine, procarbazine, prednisolone, bleomycin, etoposide, cisplatin, carboplatin, epirubicin, capecitabine, folinic acid, oxaliplatin, gemcitabine, ifosfamide, paclitaxel, and etoposide. In some embodiments, chemotherapy also includes antibody drug conjugates comprising the chemical agents or drugs described herein.

The term “complete response” refers to the disappearance of all signs of cancer in the body.

The term “partial response” refers to a decrease in the size of a tumor or in the amount of cancer in the body. The size decrease or the amount decrease is about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, 55%, 60%, 65%, 70%, 75%, 80%, 85%, 90%, 95%, or 99%.

The term “stable disease” refers to no progression of the disease after treatment.

The term “responsive to chemotherapy” refers to a patient that obtains partial response, complete response, or stable disease after treatment with chemotherapy.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary from, for example, between 1% and 15% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

Compounds of the disclosure also include crystalline and amorphous forms of the compounds, including, for example, polymorphs, pseudopolymorphs, solvates, hydrates, unsolvated polymorphs (including anhydrates), conformational polymorphs, and amorphous forms of the compound, as well as mixtures thereof. “Crystalline form” and “polymorph” are intended to include all crystalline and amorphous forms of the compounds, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

### Methods of Treating Endometrial Cancer

In one aspect, the present disclosure relates to a method of treating endometrial cancer in a human comprising the step of administering to a human in need thereof a therapeutically effective amount of a MDM2 inhibitor. In an embodiment, the cancer is responsive to chemotherapy. In an embodiment, the cancer has not progressed after chemotherapy. In an embodiment, the cancer has not progressed after treatment with a PD-1 inhibitor or a PD-L1 inhibitor. In an embodiment, the cancer has not progressed after chemotherapy and treatment with a PD-1 inhibitor or a PD-L1 inhibitor. In an embodiment, the human has advanced or recurrent endometrial cancer. In an embodiment, the human has TP53 wild-type endometrial cancer. In an embodiment, the human has cyclin-dependent kinase inhibitor 2A (CDKN2A)-deleted or inactivated endometrial cancer. In an embodiment, the human has endometrial cancer characterized by CDKN2A dysfunction. In an embodiment, the human has endometrial cancer characterized by one or more CDKN2A mutations. In an embodiment, the human has endometrial cancer characterized by loss of CDKN2A. In an embodiment, the human has endometrial cancer characterized by p161NK4a promoter hypermethylation and/or p14ARF promoter hypermethylation.

In one aspect, the present disclosure relates to a method of treating endometrial cancer comprising the step of administering to a human in need thereof a therapeutically effective amount of a MDM2 inhibitor, wherein the human is previously treated with chemotherapy and obtains a clinical benefit, wherein administration of the MDM2 inhibitor maintains the clinical benefit. In an embodiment, the clinical benefit is selected from the group consisting of objective response (OR), overall survival (OS), progression free survival (PFS), complete response (CR), partial response (PR), stable disease (SD), and combinations thereof. In an embodiment, OR is measured according to RECIST v1.1. In an embodiment, the cancer is responsive to chemotherapy. In an embodiment, the cancer has not progressed after chemotherapy. In an embodiment, the cancer has not progressed after treatment with a PD-1 inhibitor or a PD-L1 inhibitor. In an embodiment, the cancer has not progressed after chemotherapy and treatment with a PD-1 inhibitor or a PD-L1 inhibitor. In an embodiment, the human has advanced or recurrent endometrial cancer. In an embodiment, the human has TP53 wild-type endometrial cancer. In an embodiment, the human has cyclin-dependent kinase inhibitor 2A (CDKN2A)-deleted or inactivated endometrial cancer. In an embodiment, the human has endometrial cancer characterized by CDKNZA dysfunction. In an embodiment, the human has endometrial cancer characterized by one or more CDKN2A mutations. In an embodiment, the human has endometrial cancer characterized by loss of CDKN2A. In an embodiment, the human has endometrial cancer characterized by p161NK4a promoter hypermethylation and/or p14ARF promoter hypermethylation.

In some embodiments, the MDM2 inhibitor is a compound of Formula (I):

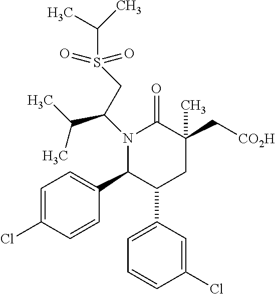 [SMILES: CC(C)[C@@H](CS(=O)(=O)C(C)C)N1C(=O)[C@@](C)(CC(=O)O)C[C@H](c2cccc(Cl)c2)[C@H]1c1ccc(Cl)cc1]

or a pharmaceutically acceptable salt thereof.

In an embodiment, the human is previously treated with chemotherapy. In an embodiment, the cancer is responsive to chemotherapy. In an embodiment, the human is previously treated with an antibody drug conjugate comprising the chemotherapy agent described herein. In an embodiment, the cancer is responsive to the antibody drug conjugate comprising the chemotherapy agent described herein In an embodiment, the human is previously treated with platinum-based therapy. In an embodiment, the cancer is responsive to platinum-based therapy.

In an embodiment, the cancer has not progressed after chemotherapy. In an embodiment, the cancer has not progressed after treatment with an antibody drug conjugate comprising the chemotherapy agent described herein. In an embodiment, the cancer has not progressed after platinum-based therapy. In an embodiment, the cancer has not progressed after treatment with a PD-1 inhibitor or a PD-L1 inhibitor. In an embodiment, the cancer has not progressed after chemotherapy and treatment with a PD-1 inhibitor or a PD-L1 inhibitor.

In an embodiment, the chemotherapy is a DNA-damaging agent. In an embodiment, the chemotherapy is a tubulin-disrupting agent. In an embodiment, the chemotherapy promotes tumor cell death. In an embodiment, the chemotherapy is an agent selected from the group consisting of cyclophosphamide, methotrexate, 5-fluorouracil, vinorelbine, doxorubicin, docetaxel, bleomycin, vinblastine, dacarbazine, mustine, vincristine, procarbazine, prednisolone, bleomycin, etoposide, cisplatin, carboplatin, epirubicin, capecitabine, folinic acid, oxaliplatin, gemcitabine, ifosfamide, etoposide, paclitaxel, and combinations thereof. In an embodiment, after initial response to chemotherapy, the response is maintained with a compound of Formula (I). In an embodiment, the initial response is a complete response. In an embodiment, the initial response is a partial response.

In an embodiment, the PD-1 inhibitor is an anti-PD-1 antibody. In an embodiment, the anti-PD-1 antibody is selected from group consisting of nivolumab, pembrolizumab, pidilizumab, cemiplimab-rwlc, AMP-224, AMP-514, PDR001, ezabenlimab, dostarlimab, retifanlimab, and fragments, conjugates, biosimilars, or variants thereof. In an embodiment, after initial response to a PD-1 inhibitor, the response is maintained with a compound of Formula (I). In an embodiment, the initial response is a complete response. In an embodiment, the initial response is a partial response.

In an embodiment, the PD-L1 inhibitor is an anti-PD-L1 antibody. In an embodiment, the anti-PD-L1 antibody is selected from the group consisting of BMS-936559, durvalumab, atezolizumab, avelumab, MPDL3280A, MED14736, MSB0010718C, MDX1105-01, and fragments, conjugates, biosimilars, or variants thereof. In an embodiment, after initial response to a PD-L1 inhibitor, the response is maintained with a compound of Formula (I). In an embodiment, the initial response is a complete response. In an embodiment, the initial response is a partial response.

In an embodiment, the human is previously treated with radiation therapy. In an embodiment, the cancer is responsive to radiation therapy. In an embodiment, the cancer has not progressed after radiation therapy. In an embodiment, after initial response to radiation therapy, the response is maintained with a compound of Formula (I). In an embodiment, the initial response is a complete response. In an embodiment, the initial response is a partial response.

In an embodiment, the human received an induction treatment with chemotherapy. In an embodiment, the human received an induction treatment with immunotherapy. In an embodiment, the human received an induction treatment with a combination of chemotherapy and immunotherapy. In an embodiment, the immunotherapy in the induction treatment comprises treatment with a PD-1 inhibitor. In an embodiment, the immunotherapy in the induction treatment comprises treatment with a PD-L1 inhibitor. In an embodiment, the human achieved a clinical benefit from the induction treatment.

In an embodiment, the MDM2 inhibitor is a pharmaceutically acceptable salt of a compound of Formula (I).

In some embodiments, the compound of Formula (I) is in a crystalline form. In an embodiment, the crystalline form of the compound of Formula (I) is characterized by a powder X-ray diffraction pattern comprising at least three peaks at diffraction angle 2 theta degrees selected from a group consisting of peaks at approximately 11.6, 12.4, 18.6, 19.0, 21.6 and 23.6±0.1.

In some embodiments, the compound of Formula (I) is in an amorphous form.

In some embodiments, the compound of Formula (I) is in a free form.

In an embodiment, the compound of Formula (I) is administered once daily at a dose selected from the group consisting of about 15 mg, about 25 mg, about 30 mg, about 50 mg, about 60 mg, about 75 mg, about 90 mg, about 100 mg, about 120 mg, about 150 mg, about 175 mg, about 180 mg, about 200 mg, about 225 mg, about 240 mg, about 250 mg, about 275 mg, about 300 mg, about 325 mg, about 350 mg, about 360 mg, about 375 mg, and about 480 mg. In an embodiment, the compound of Formula (I) is administered twice daily at a dose selected from the group consisting of about 15 mg, about 25 mg, about 30 mg, about 50 mg, about 60 mg, about 75 mg, about 90 mg, about 100 mg, about 120 mg, about 150 mg, about 175 mg, about 180 mg, about 200 mg, about 225 mg, about 240 mg, about 250 mg, about 275 mg, about 300 mg, about 325 mg, about 350 mg, about 360 mg, about 375 mg, and about 480 mg.

In an embodiment, the human is treated with the compound of Formula (I) for a period selected from the group consisting of about 7 days, about 14 days, about 21 days, about 28 days, about 35 days, about 42 days, about 49 days, and about 56 days.

In an embodiment, the compound of Formula (I) is orally administered.

In an embodiment, the therapeutically effective amount of the MDM2 inhibitor is about 180 mg. In an embodiment, the therapeutically effective amount of the MDM2 inhibitor is about 240 mg.

In an embodiment, the human is previously treated with platinum-based therapy. In an embodiment, the human is previously treated with radiation therapy.

In an embodiment, the MDM2 inhibitor is administered intermittently. In an embodiment, the MDM2 inhibitor is administered on days 1-7, followed by discontinuance of administration for days 8-28 on a 28-day cycle, optionally repeating the cycle of administration and discontinuation of administration for 2, 3, 4, 5, 6, 7, 8, 9, or 10 times.

In an embodiment, the MDM2 inhibitor is administered in a dosage selected from the group consisting of about 15 mg QD, about 25 mg QD, about 30 mg QD, about 50 mg QD, about 60 mg QD, about 75 mg QD, about 90 mg QD, about 100 mg QD, about 120 mg QD, about 125 mg QD, about 150 mg QD, about 175 mg QD, about 180 mg QD, about 200 mg QD, about 225 mg QD, about 240 mg QD, about 250 mg QD, about 275 mg QD, about 300 mg QD, about 325 mg QD, about 350 mg QD, about 360 mg QD, about 375 mg QD, about 480 mg QD, about 15 mg BID, about 25 mg BID, about 30 mg BID, about 50 mg BID, about 60 mg BID, about 75 mg BID, about 90 mg BID, about 100 mg BID, about 120 mg BID, about 125 mg BID, about 150 mg BID, about 175 mg BID, about 180 mg BID, about 200 mg BID, about 225 mg BID, about 240 mg BID, about 250 mg BID, about 275 mg BID, about 300 mg BID, about 325 mg BID, about 350 mg BID, about 360 mg BID, about 375 mg BID, and about 480 mg BID. In an embodiment, the MDM2 inhibitor is administered to a human according to the Section “Dosages and Dosing Regimens.”

### MDM2 Inhibitors

The compound of Formula (I) has the structure and name shown below.

### 2-((3R,5R,6S)-5-(3-chlorophenyl)-6-(4-chlorophenyl)-1-((S)-1-(isopropylsulfonyl)-3-methylbutan-2-yl)-3-methyl-2-oxopiperidin-3-yl) acetic Acid

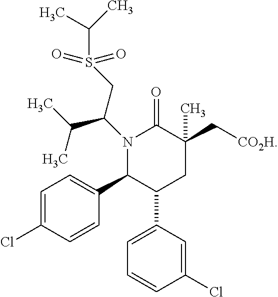

The synthesis of the compound of Formula (I) is set forth in WO2011153509 and WO2014200937; U.S. Pat. Nos. 8,569,341; 9,593,129; 9,296,736; 9,623,018; 9,757,367; 9,801,867; 9,376,386; and 9,855,259, the disclosures of which are incorporated by reference herein in their entirety.

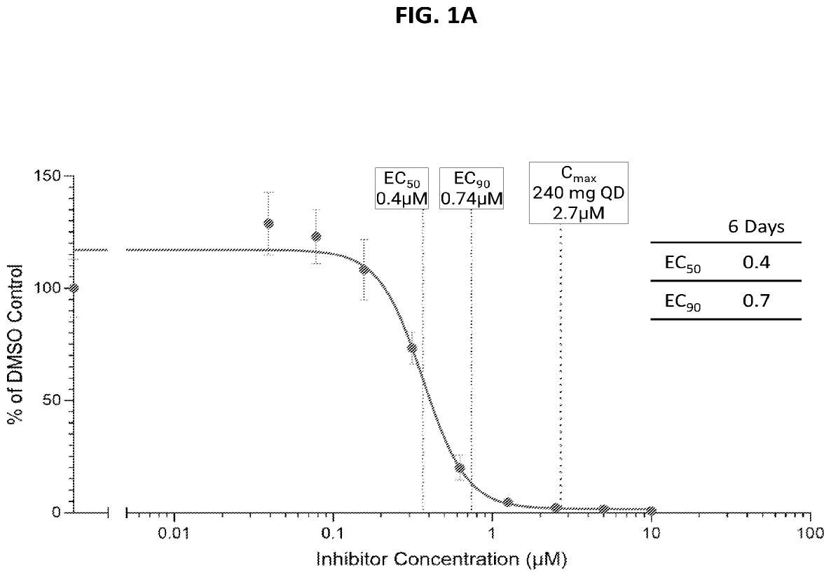

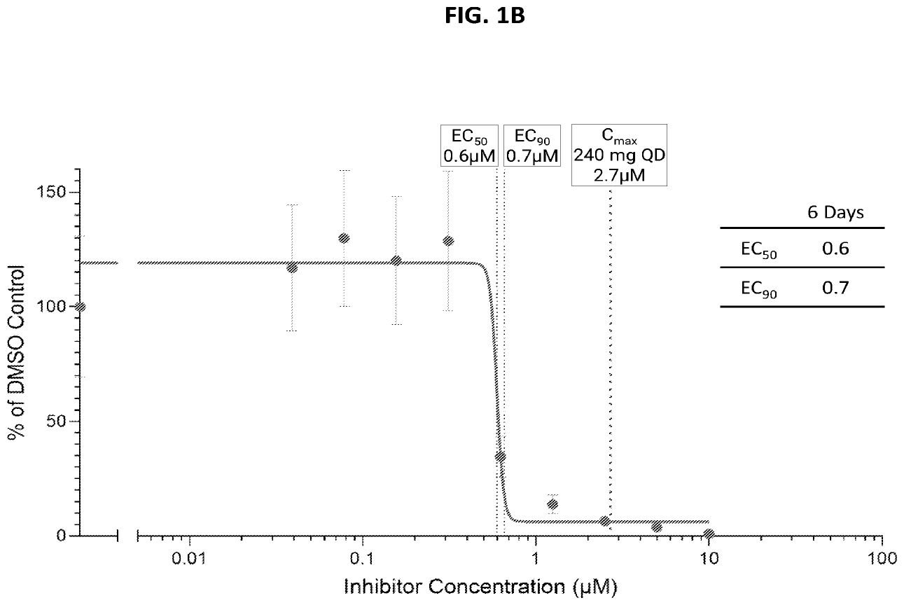

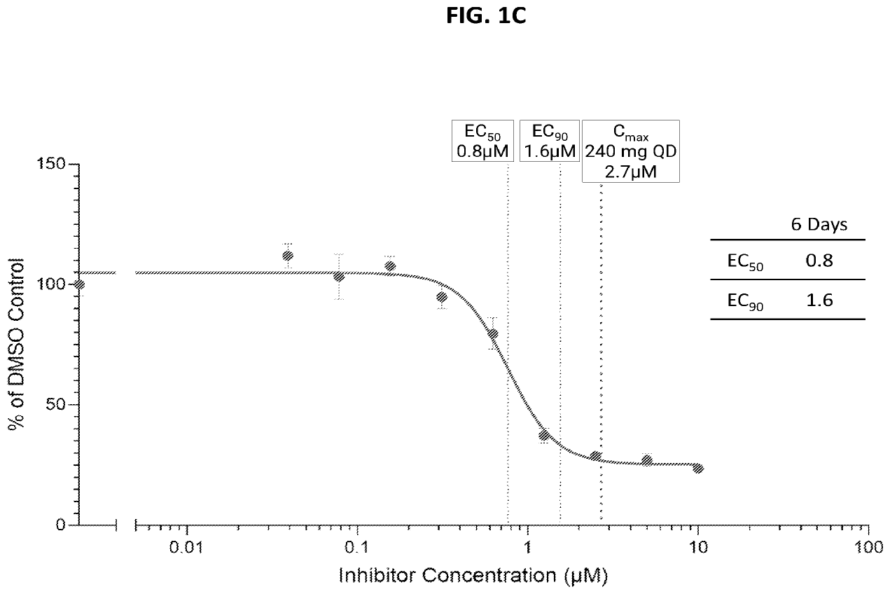

In an embodiment, the compound of Formula (I) is in an amorphous form. In an embodiment, the MDM2 inhibitor is the compound of Formula (I) in a crystalline form. In an embodiment, the MDM2 inhibitor is the compound of Formula (I) in a crystalline anhydrous form. In an embodiment, the MDM2 inhibitor is the compound of Formula (I) in a crystalline anhydrous form characterized by a powder X-ray diffraction pattern comprising peaks at diffraction angle 2 theta degrees at approximately 11.6, 12.4, 18.6, 19.0, 21.6 and 23.6. In an embodiment, the MDM2 inhibitor is the compound of Formula (I) in a crystalline anhydrous form having the X-ray diffraction pattern substantially shown in FIG. 1 of WO2014/200937. The method of making such crystalline form was disclosed in the International Application WO2014/200937, the disclosure of which is incorporated herein by reference in its entirety.

In an embodiment, the MDM2 inhibitor is 4-(2-((3R,5R,6S)-1-((S)-2-(tert-butylsulfonyl)-1-cyclopropylethyl)-6-(4-chloro-3-fluorophenyl)-5-(3-chlorophenyl)-3-methyl-2-oxopiperidin-3-yl)acetamido)-2-methoxybenzoic acid, referred to herein as the compound of Formula (II). The synthesis of the compound of Formula (II) is set forth in U.S. Pat. No. 8,952,036, the disclosure of which is incorporated by reference herein in its entirety.

In an embodiment, the MDM2 inhibitor is RG7388. RG7388 is also known as 4-[[(2R,3S,4R,5S)-3-(3-chloro-2-fluorophenyl)-4-(4-chloro-2-fluorophenyl)-4-cyano-5-(2,2-dimethyl propyl)pyrrolidine-2-carbonyl]amino]-3-methoxybenzoic acid.

In an embodiment, the MDM2 inhibitor is triptolide. Triptolide is also known as (5bS,6aS,7aS,8R,8aR,9aS,9bS,10aS,10bS)-8-hydroxy-8a-isopropyl-10b-methyl-2,5,5b,6,6a,8,8a,9a,9b,10b-decahydrotris(oxireno) [2′,3′:4b,5; 2″,3″:6,7; 2′″,3′″:8a,9]phenanthro[1,2-c]furan-3(1H)-one.

In an embodiment, the MDM2 inhibitor is Nutlin-3a. Nutlin-3a is also known as 4-[(4S,5R)-4,5-bis(4-chlorophenyl)-2-(4-methoxy-2-propan-2-yloxyphenyl)-4,5-dihydroimidazole-1-carbonyl]piperazin-2-one.

In an embodiment, the MDM2 inhibitor is HDM201. HDM201 is also known as (4S)-5-(5-chloro-1-methyl-2-oxopyridin-3-yl)-4-(4-chlorophenyl)-2-(2,4-dimethoxypyrimidin-5-yl)-3-propan-2-yl-4H-pyrrolo[3,4-d]imidazol-6-one.

In an embodiment, the MDM2 inhibitor is RG7112. RG7112 is also known as [(4S,5R)-2-(4-tert-butyl-2-ethoxyphenyl)-4,5-bis(4-chlorophenyl)-4,5-dimethylimidazol-1-yl]-[4-(3-methylsulfonylpropyl)piperazin-1-yl]methanone.

In an embodiment, the MDM2 inhibitor is CGM097A. CGM097A has is also known as (1S)-1-(4-chlorophenyl)-6-methoxy-2-[4-[methyl-[[4-(4-methyl-3-oxopiperazin-1-yl)cyclohexyl]methyl]amino]phenyl]-7-propan-2-yloxy-1,4-dihydroisoquinolin-3-one.

In an embodiment, the MDM2 inhibitor is nutlin-3. Nutlin-3 is also known as 4-[4,5-bis(4-chlorophenyl)-2-(4-methoxy-2-propan-2-yloxyphenyl)-4,5-dihydroimidazole-1-carbonyl]piperazin-2-one.

In an embodiment, the MDM2 inhibitor is SJ-172550. SJ-172550 is known as methyl 2-[2-chloro-6-ethoxy-4-[(3-methyl-5-oxo-1-phenylpyrazol-4-ylidene)methyl]phenoxy]acetate.

In an embodiment, the MDM2 inhibitor is SAR405838. SAR405838 is also known as (2′R,3R,3′S,5′S)-6-chloro-3′-(3-chloro-2-fluorophenyl)-5′-(2,2-dimethylpropyl)-N-(4-hydroxycyclohexyl)-2-oxospiro[1H-indole-3,4′-pyrrolidine]-2′-carboxamide.

In an embodiment, the MDM2 inhibitor is MI-773. MI-773 is known as (2′R,3S,3′S,5′R)-6-chloro-3′-(3-chloro-2-fluorophenyl)-5′-(2,2-dimethylpropyl)-N-(4-hydroxycyclohexyl)-2-oxospiro[1H-indole-3,4′-pyrrolidine]-2′-carboxamide.

In an embodiment, the MDM2 inhibitor is MX69. MX69 is known as 4-[8-[(3,4-dimethylphenyl)sulfamoyl]-3a,4,5,9b-tetrahydro-3H-cyclopenta[c]quinolin-4-yl]benzoic acid.

In an embodiment, the MDM2 inhibitor is YH239-EE. YH239-EE is known as ethyl 3-[2-(tert-butylamino)-1-[(4-chlorophenyl)methyl-formylamino]-2-oxoethyl]-6-chloro-1H-indole-2-carboxylate.

In an embodiment, the MDM2 inhibitor is R08994. R08994 is known as (2′R,3R,3′S,5′S)—N-(4-carbamoyl-2-methoxyphenyl)-6-chloro-3′-(3-chloro-2-fluorophenyl)-5′-(2,2-dimethylpropyl)-2-oxospiro[1H-indole-3,4′-pyrrolidine]-2′-carboxamide.

In an embodiment, the MDM2 inhibitor is nutlin-3b. Nutlin-3b is known as 4-[(4R,5S)-4,5-bis(4-chlorophenyl)-2-(4-methoxy-2-propan-2-yloxyphenyl)-4,5-dihydroimidazole-1-carbonyl]piperazin-2-one.

In an embodiment, the MDM2 inhibitor is Serdemetan. Serdemetan is known as 1-N-[2-(1H-indol-3-yl)ethyl]-4-N-pyridin-4-ylbenzene-1,4-diamine.

In an embodiment, the MDM2 inhibitor is NSC59984. NSC59984 is known as (E)-1-(4-methylpiperazin-1-yl)-3-(5-nitrofuran-2-yl)prop-2-en-1-one.

In an embodiment, the MDM2 inhibitor is CHEMBL2386350. CHEMBL2386350 is known as 2-[4-[(4S,5R)-2-(4-tert-butyl-2-ethoxyphenyl)-4,5-bis(4-chlorophenyl)-4,5-dimethylimidazole-1-carbonyl]piperazin-1-yl]-1-morpholin-4-ylethanone.

In an embodiment, the MDM2 inhibitor is CGM0970B. CGM0970B is known as (1R)-1-(4-chlorophenyl)-6-methoxy-2-[4-[methyl-[[4-(4-methyl-3-oxopiperazin-1-yl)cyclohexyl]methyl]amino]phenyl]-7-propan-2-yloxy-1,4-dihydroisoquinolin-3-one.

In an embodiment, the MDM2 inhibitor is MK-8242. MK-8242 is known as 4-amino-1-[(2R,3S,4S,5R)-3,4-dihydroxy-5-(hydroxymethyl)oxolan-2-yl]pyrimidin-2-one.

In an embodiment, the MDM2 inhibitor is DS-3032. DS-3032 is known as (3′R,4′S,5′R)—N-((3R,6S)-6-carbamoyltetrahydro-2H-pyran-3-yl)-6″-chloro-4′-(2-chloro-3-fluoropyridin-4-yl)-4,4-dimethyl-2″-oxodispiro[cyclohexane-1,2′-pyrrolidine-3′,3″-indoline]-5′-carboxamide.

In an embodiment, the MDM2 inhibitor is DS-3032B. DS-3032B is known as (3′R,4′S,5′R)—N-((3R,6S)-6-carbamoyltetrahydro-2H-pyran-3-yl)-6″-chloro-4′-(2-chloro-3-fluoropyridin-4-yl)-4,4-dimethyl-2″-oxodispiro[cyclohexane-1,2′-pyrrolidine-3′,3″-indoline]-5′-carboxamide 4-methylbenzenesulfonate.

In an embodiment, the MDM2 inhibitor is APG-115. APG-115 is known as 4-((3′R,4′S,5′R)-6″-Chloro-4′-(3-chloro-2-fluorophenyl)-1′-ethyl-2″-oxodispiro[cyclohexane-1,2′-pyrrolidine-3′,3″-indoline]-5′-carboxamido)bicyclo[2.2.2]octane-1-carboxylic acid.

In an embodiment, the MDM2 inhibitor is APG-115. APG-115 is known as 4-((3′R,4′S,5′R)-6″-chloro-4′-(3-chloro-2-fluorophenyl)-2″-oxodispiro[cyclohexane-1,2′-pyrrolidine-3′,3″-indoline]-5′-carboxamido)benzoic acid.

In an embodiment, the MDM2 inhibitor is Milademetan (also known as DS-3032).

Combination with Immunotherapy

In an embodiment, the methods further comprise administration of an immunotherapy in combination with the MDM2 inhibitor. In an embodiment, the immunotherapy is a PD-1 inhibitor or a PD-L1 inhibitor.

In an embodiment, the PD-1 inhibitor is an anti-PD-1 antibody. In an embodiment, the anti-PD-1 antibody is selected from group consisting of nivolumab, pembrolizumab, pidilizumab, cemiplimab-rwlc, AMP-224, AMP-514, PDR001, ezabenlimab, dostarlimab, retifanlimab, and fragments, conjugates, biosimilars, or variants thereof. In an embodiment, after initial response to a PD-1 inhibitor, the response is maintained with a compound of Formula (I) in combination with a PD-1 inhibitor. In an embodiment, the initial response is a complete response. In an embodiment, the initial response is a partial response.

In an embodiment, the PD-L1 inhibitor is an anti-PD-L1 antibody. In an embodiment, the anti-PD-L1 antibody is selected from the group consisting of BMS-936559, durvalumab, atezolizumab, avelumab, MPDL3280A, MED14736, MSB0010718C, MDX1105-01, and fragments, conjugates, biosimilars, or variants thereof. In an embodiment, after initial response to a PD-L1 inhibitor, the response is maintained with a compound of Formula (I) in combination with a PD-L1 inhibitor. In an embodiment, the initial response is a complete response. In an embodiment, the initial response is a partial response.

### Pharmaceutical Compositions

In some embodiments, the disclosure provides pharmaceutical compositions comprising a MDM2 inhibitor or a pharmaceutically acceptable salt thereof for treating endometrial cancer in a human subject. In some embodiments, the disclosure provides pharmaceutical compositions comprising a MDM2 inhibitor or a pharmaceutically acceptable salt thereof for treating endometrial cancer in a human subject, wherein the human subject is previously treated with chemotherapy and obtains a clinical benefit, wherein the MDM2 inhibitor maintains the clinical benefit.

The pharmaceutical compositions are typically formulated to provide a therapeutically effective amount of a MDM2 inhibitor or a pharmaceutically acceptable salt thereof. Where desired, the pharmaceutical compositions contain a pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants. Where desired, other ingredients in addition to a MDM2 inhibitor or a pharmaceutically acceptable salt thereof may be mixed into a preparation or both components may be formulated into separate preparations for use in combination separately or at the same time.

In selected embodiments, the concentration of a MDM2 inhibitor or a pharmaceutically acceptable salt thereof provided in the pharmaceutical compositions of the disclosure is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v.

In selected embodiments, the concentration of a MDM2 inhibitor or a pharmaceutically acceptable salt thereof provided in the pharmaceutical compositions of the disclosure is independently greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v.

In selected embodiments, the concentration of a MDM2 inhibitor or a pharmaceutically acceptable salt thereof is independently in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12% or approximately 1% to approximately 10% w/w, w/v or v/v.

In selected embodiments, the concentration of a MDM2 inhibitor or a pharmaceutically acceptable salt thereof is independently in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In selected embodiments, the amount of a MDM2 inhibitor or a pharmaceutically acceptable salt thereof is independently equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g or 0.0001 g.

In selected embodiments, the amount of a MDM2 inhibitor or a pharmaceutically acceptable salt thereof is independently more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g or 10 g.

A MDM2 inhibitor or a pharmaceutically acceptable salt thereof are effective over a wide dosage range. For example, in the treatment of adult humans, dosages independently ranging from 0.01 to 1000 mg, from 0.5 to 100 mg, from 1 to 50 mg per day, and from 5 to 40 mg per day are examples of dosages that may be used. The exact dosage will depend upon the route of administration, the form in which the compound is administered, the gender and age of the subject to be treated, the body weight of the subject to be treated, and the preference and experience of the attending physician.

Described below are non-limiting exemplary pharmaceutical compositions and methods for preparing the same.

### Pharmaceutical Compositions for Oral Administration

In selected embodiments, the disclosure provides a pharmaceutical composition for oral administration comprising a MDM2 inhibitor or a pharmaceutically acceptable salt thereof, and a pharmaceutical excipient suitable for oral administration.

In selected embodiments, the disclosure provides a solid pharmaceutical composition for oral administration containing: (i) an effective amount of a MDM2 inhibitor or a pharmaceutically acceptable salt thereof, in combination and (ii) a pharmaceutical excipient suitable for oral administration. In selected embodiments, the composition further contains (iii) an effective amount of at least one additional active ingredient.

In selected embodiments, the pharmaceutical composition may be a liquid pharmaceutical composition suitable for oral consumption. Pharmaceutical compositions of the disclosure suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods, but all methods include the step of bringing the active ingredient(s) into association with the carrier, which constitutes one or more necessary ingredients. In general, the compositions are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The disclosure further encompasses anhydrous pharmaceutical compositions and dosage forms since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the disclosure can be prepared using anhydrous or low moisture containing ingredients and low moisture or low humidity conditions. Pharmaceutical compositions and dosage forms of the disclosure which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

A MDM2 inhibitor or a pharmaceutically acceptable salt thereof can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the disclosure to provide tablets that disintegrate when exposed to an aqueous environment. Too much of a disintegrant may produce tablets which disintegrate in the bottle. Too little may be insufficient for disintegration to occur, thus altering the rate and extent of release of the active ingredients from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the disclosure include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the disclosure include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the essential active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants which can be used to form pharmaceutical compositions and dosage forms of the disclosure include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In an embodiment, the composition may include a solubilizer to ensure good solubilization and/or dissolution of the compound of the present disclosure and to minimize precipitation of the compound of the present disclosure. This can be especially important for compositions for non-oral use, such as for compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, epsilon-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals and alkaline earth metals. Examples may include, but are not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid.

### Pharmaceutical Compositions for Injection

In selected embodiments, the disclosure provides a pharmaceutical composition for injection comprising a MDM2 inhibitor or a pharmaceutically acceptable salt thereof, and a pharmaceutical excipient suitable for injection. Components and amounts of agents in the compositions are as described herein.

The forms in which the compositions of the present disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol and liquid polyethylene glycol (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid and thimerosal.

Sterile injectable solutions are prepared by incorporating a MDM2 inhibitor or a pharmaceutically acceptable salt thereof in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

Administration of a MDM2 inhibitor or a pharmaceutically acceptable salt thereof, or pharmaceutical composition of the compound can be effected by any method that enables delivery of the compound to the site of action. These methods include oral routes, intraduodenal routes, parenteral injection (including intravenous, intra-arterial, subcutaneous, intramuscular, intravascular, intraperitoneal or infusion), topical (e.g., transdermal application), rectal administration, via local delivery by catheter or stent or through inhalation. The compound can also be administered intraadiposally or intrathecally.

Exemplary parenteral administration forms include solutions or suspensions of active compound in sterile aqueous solutions, for example, aqueous propylene glycol or dextrose solutions. Such dosage forms can be suitably buffered, if desired.

The disclosure also provides kits. The kits include a MDM2 inhibitor or a pharmaceutically acceptable salt thereof, either alone or in combination in suitable packaging, and written material that can include instructions for use, discussion of clinical studies and listing of side effects. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another active pharmaceutical ingredient. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in selected embodiments, be marketed directly to the consumer. In an embodiment, the disclosure provides a kit comprising a MDM2 inhibitor or a pharmaceutically acceptable salt thereof for use in the treatment of endometrial cancer.

### Dosages and Dosing Regimens

The amount administered of a MDM2 inhibitor or a pharmaceutically acceptable salt thereof will be dependent on the human being treated, the severity of the disorder or condition, the rate of administration, the disposition of the compounds and the discretion of the prescribing physician. However, an effective dosage is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect—e.g., by dividing such larger doses into several small doses for administration throughout the day.

In some embodiments, a MDM2 inhibitor or a pharmaceutically acceptable salt thereof is administered in a single dose. Typically, such administration will be by injection—e.g., intravenous injection, in order to introduce the agents quickly. However, other routes may be used as appropriate.

In some embodiments, a MDM2 inhibitor or a pharmaceutically acceptable salt thereof is administered in multiple doses for treating endometrial cancer.

In an embodiment, a MDM2 inhibitor or a pharmaceutically acceptable salt thereof is administered in multiple doses. In an embodiment, a MDM2 inhibitor or a pharmaceutically acceptable salt thereof is administered in multiple doses by injection—e.g., intravenous injection. In an embodiment, dosing may be once, twice, three times, four times, five times, six times, or more than six times per day. In an embodiment, dosing may be selected from the group consisting of once a day, twice a day, three times a day, four times a day, five times a day, six times a day, once every other day, once weekly, twice weekly, three times weekly, four times weekly, biweekly, and monthly. In other embodiments, a MDM2 inhibitor or a pharmaceutically acceptable salt thereof is administered about once per day to about six times per day. In some embodiments a MDM2 inhibitor or a pharmaceutically acceptable salt thereof is administered once daily, while in other embodiments a MDM2 inhibitor or a pharmaceutically acceptable salt thereof is administered twice daily, and in other embodiments a MDM2 inhibitor or a pharmaceutically acceptable salt thereof is administered three times daily. In some embodiments a MDM2 inhibitor or a pharmaceutically acceptable salt thereof is administered three times a week, including every Monday, Wednesday, and Friday.

Administration of a MDM2 inhibitor or a pharmaceutically acceptable salt thereof may continue as long as necessary. In some embodiments, a MDM2 inhibitor or a pharmaceutically acceptable salt thereof is administered for more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or more days. In some embodiments, a MDM2 inhibitor or a pharmaceutically acceptable salt thereof is administered for about 14 days, about 21 days, about 28 days, about 35 days, about 42 days, about 49 days, or about 56 days. In some embodiments, a MDM2 inhibitor or a pharmaceutically acceptable salt thereof is administered chronically on an ongoing basis—e.g., for the treatment of chronic effects. In another embodiment the administration of a MDM2 inhibitor or a pharmaceutically acceptable salt thereof continues for less than about 7 days. In yet another embodiment the administration continues for more than about 6, 10, 14, 28 days, two months, three months, four months, five months, six months, seven months, eight months, nine months, ten months, eleven months or one year. In some embodiments, the administration continues for more than about one year, two years, three years, four years, or five years. In some embodiments, continuous dosing is achieved and maintained as long as necessary.

In some embodiments, an effective dosage of a MDM2 inhibitor or a pharmaceutically acceptable salt thereof is in the range of about 1 mg to about 500 mg, about 10 mg to about 300 mg, about 20 mg to about 250 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 202 mg. In some embodiments, an effective dosage of a MDM2 inhibitor or a pharmaceutically acceptable salt thereof is about 15 mg, about 25 mg, about 30 mg, about 50 mg, about 60 mg, about 75 mg, about 90 mg, about 100 mg, about 120 mg, about 125 mg, about 150 mg, about 175 mg, about 180 mg, about 200 mg, about 225 mg, about 240 mg, about 250 mg, about 275 mg, about 300 mg, about 325 mg, about 350 mg, about 360 mg, about 375 mg, about 400 mg, about 425 mg, about 450 mg, about 475 mg, about 480 mg, or about 500 mg. In some embodiments, an effective dosage of a MDM2 inhibitor or a pharmaceutically acceptable salt thereof is 15 mg, 25 mg, 30 mg, 50 mg, 60 mg, 75 mg, 90 mg, 100 mg, 120 mg, 125 mg, 150 mg, 175 mg, 180 mg, 200 mg, 225 mg, 240 mg, 250 mg, 275 mg, 300 mg, 325 mg, 350 mg, 360 mg, 375 mg, and 480 mg.

In some embodiments, an effective dosage of a MDM2 inhibitor or a pharmaceutically acceptable salt thereof is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg. In some embodiments, an effective dosage of a MDM2 inhibitor or a pharmaceutically acceptable salt thereof is about 0.35 mg/kg, about 0.7 mg/kg, about 1 mg/kg, about 1.4 mg/kg, about 1.8 mg/kg, about 2.1 mg/kg, about 2.5 mg/kg, about 2.85 mg/kg, about 3.2 mg/kg, or about 3.6 mg/kg.

In some embodiments, a MDM2 inhibitor or a pharmaceutically acceptable salt thereof is administered at a dosage of 10 to 500 mg BID, including a dosage of 15 mg, 25 mg, 30 mg, 50 mg, 60 mg, 75 mg, 90 mg, 100 mg, 120 mg, 125 mg, 150 mg, 175 mg, 180 mg, 200 mg, 225 mg, 240 mg, 250 mg, 275 mg, 300 mg, 325 mg, 350 mg, 360 mg, 375 mg, and 480 mg BID.

In some embodiments, a MDM2 inhibitor or a pharmaceutically acceptable salt thereof is administered at a dosage of 10 to 500 mg QD, including a dosage of 15 mg, 25 mg, 30 mg, 50 mg, 60 mg, 75 mg, 90 mg, 100 mg, 120 mg, 125 mg, 150 mg, 175 mg, 180 mg, 200 mg, 225 mg, 240 mg, 250 mg, 275 mg, 300 mg, 325 mg, 350 mg, 360 mg, 375 mg, and 480 mg QD.

An effective amount of a MDM2 inhibitor or a pharmaceutically acceptable salt thereof may be administered in either single or multiple doses by any of the accepted modes of administration of agents having similar utilities, including buccal, sublingual, and transdermal routes, by intra-arterial injection, intravenously, parenterally, intramuscularly, subcutaneously or orally.

In some embodiments, a MDM2 inhibitor or a pharmaceutically acceptable salt thereof is administered to a subject intermittently, known as intermittent administration. By “intermittent administration”, it is meant a period of administration of a therapeutically effective dose of a MDM2 inhibitor or a pharmaceutically acceptable salt thereof, followed by a time period of discontinuance, which is then optionally followed by another administration period, optionally followed by another time period of discontinuance and so on. In each administration period, the dosing frequency can be independently select from three times daily, twice daily, daily, once weekly, twice weekly, three times weekly, four times weekly, five times weekly, six times weekly or monthly. In an embodiment, the MDM2 inhibitor is the compound of Formula (I).

By “period of discontinuance” or “discontinuance period” or “rest period”, it is meant the length of time when discontinuing the administration of a MDM2 inhibitor or a pharmaceutically acceptable salt thereof. The time period of discontinuance may be longer or shorter than the administration period or the same as the administration period. During the discontinuance period, other therapeutic agents other than a MDM2 inhibitor or a pharmaceutically acceptable salt thereof may be administered. The term “cycle” or “treatment cycle” refers to the combination of the time period of administration together with the time period of discontinuance. For example, a 28-day cycle with days 1-7 on and days 8-28 off refers to days 1-7 are the time period of administration and days 8-28 are the time period of discontinuance. As another example, a 28-day cycle with days 1-5 on and days 6-28 off refers to days 1-5 are the time period of administration and days 6-28 are the time period of discontinuance.

In an embodiment, a MDM2 inhibitor or a pharmaceutically acceptable salt thereof is administered to a human subject in need thereof for treating endometrial cancer for a first administration period, then followed by a first discontinuance period, then optionally followed by a second administration period, then optionally followed by a second discontinuance period, and so on. In some embodiments, this pattern of administration and discontinuation of administration is repeated for a sufficient duration to treat the endometrial cancer. Such duration may be one week, two weeks, three weeks, four weeks, one month, five weeks, six weeks, seven weeks, two months, nine weeks, ten weeks, eleven weeks, three months, thirteen weeks, fourteen weeks, fifteen weeks, four months, five months, six months, seven months, or eight months.

The first administration period, the second administration period, the first discontinuance period and the second discontinuance period are each independently selected from the group consisting of more than 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, one month, five weeks, six weeks, seven weeks, two months, nine weeks, ten weeks, eleven weeks, three months, thirteen weeks, fourteen weeks, fifteen weeks, four months, and more days, in which a MDM2 inhibitor or a pharmaceutically acceptable salt thereof is administered to a subject three times daily, twice daily, daily, once weekly, twice weekly, three times weekly, four times weekly, five times weekly, six times weekly or monthly. In an embodiment, the first administration period is at same length as the second administration period. In an embodiment, the first administration period is shorter than the second administration period. In an embodiment, the first administration period is longer than the second administration period. In an embodiment, the first administration period is about 7 days and the first discontinuance period is about 14 days. In an embodiment, the first administration period is about 5 days and the first discontinuance period is about 23 days. In an embodiment, the first administration period is about 7 days and the first discontinuance period is about 21 days. In an embodiment, the first administration period is about 7 days; the second administration period is about 7 days; the first discontinuance period is about 14 days; and the second discontinuance period is about 14 days. In an embodiment, the first administration period is about 5 days; the second administration period is about 5 days; the first discontinuance period is about 23 days; and the second discontinuance period is about 23 days. In an embodiment, the first administration period is about 7 days; the second administration period is about 7 days; the first discontinuance period is about 21 days; and the second discontinuance period is about 21 days. In some embodiments, the treatment period is one, two, three, four, five, six, seven, eight, nine or ten cycles.

In an embodiment, the MDM2 inhibitor is the compound of Formula (I).

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1: Inhibition of Endometrial Cancer Cell Growth

The effect of MDM2 inhibitor compound of Formula (I) on growth of a p53 wild-type (TP53 WT) endometrial cancer cell line was assessed in HEC-151 cells, HEC-265 cells, and HEC-108 cells as described below.

Test and Control Article Preparation: A stock solution for Formula (I) was created by dissolving the compound in 100% DMSO to a concentration of 10 mM which was stored at −20 °C. Working solutions ranging from 0.04 μM to 10 μM to generate a 10-point dilution series were prepared by diluting the stock solution in culture media specific to each cell line (cell line assay media). Final concentrations of DMSO were kept constant at 0.1% to maintain cell viability.

The vehicle control was prepared by diluting 100% DMSO in cell line assay media to a final concentration of 0.1%, which is consistent with the final DMSO concentration of the test article.

Treatment of Cells for Proliferation Assay: Cells were counted (Countess III automated cell counter, Thermo Fisher Scientific) using a cell viability imaging kit (Thermo Fisher Scientific; cat #R37609). Cells were plated in triplicate at 1.5×10² to 2.5×10³ cells per well, depending on cell line and treatment duration, in a 96-well clear-bottom white-wall plate and left to adhere overnight at 37 °C with 5% CO₂ (Panasonic MCO-170AICUVL-PA incubator, PHC Corporation of North America). The following day (Day 0), cells were treated with 0.1% DMSO and a nine-point 2-fold titration of Formula (I) from 0.04 μM to 10 μM. The cells were left to incubate for 3 or 6 days at 37 °C with 5% CO₂.

On Day 0 prior treatment, and after the treatment period, the number of viable cells were measured using the CellTiter-Glo® Luminescent Cell Viability Assay (Promega) according to manufacturer instructions. The luminescence for each treatment condition was normalized to the DMSO control as a percent of control. Dose response curves were generated and EC50s and EC90s were determined based on percent of DMSO control. Additionally, the luminescence for each treatment condition was compared to the luminescence acquired on Day 0 and the amount of cell growth and/or cell death was determined. Growth inhibition was calculated on a 200-point scale according to published equations (Canon, Mol Cancer Ther. 2015; 14(3):649-658) where luminescence for each treatment condition was normalized to the DMSO control and compared to the luminescence of time zero. Values of 0, 100 and 200 represented uninhibited cell growth (DMSO control), cell stasis, and complete cell killing, respectively.

Statistical Analysis: Dose response curves were generated and EC50s and EC90s were calculated using GraphPad Prism v9 (GraphPad Software, San Diego, CA). The results are shown in FIG. 1 and demonstrate growth inhibition by the compound of Formula (I) at clinically relevant doses. Specifically, after the 6-day incubation the EC₅₀ values were 0.4 μM for HEC-151 cells and 0.6 μM for HEC-265 cells. Near complete growth inhibition (EC₉₀) was at 0.7 μM for both HEC-151 and HEC-265 cells. The HEC-108 cell line was less sensitive to Formula (I)-induced cell killing and only nearly reached cell stasis at 10 μM after a 6-day exposure.

1. A method of treating endometrial cancer comprising the step of administering to a human in need thereof a therapeutically effective amount of a MDM2 inhibitor, wherein the human is previously treated with chemotherapy and obtains a clinical benefit, wherein administration of the MDM2 inhibitor maintains the clinical benefit, wherein the MDM2 inhibitor is a compound of Formula (I): 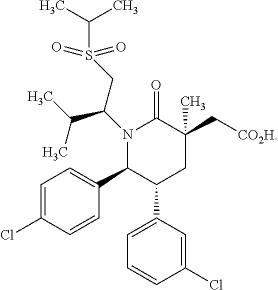 [SMILES: CC(C)[C@@H](CS(=O)(=O)C(C)C)N1C(=O)[C@@](C)(CC(=O)O)C[C@H](c2cccc(Cl)c2)[C@H]1c1ccc(Cl)cc1]
or a pharmaceutically acceptable salt thereof.

2. The method of claim 1, wherein the clinical benefit is selected from the group consisting of objective response (OR), overall survival (OS), progression free survival (PFS), complete response (CR), partial response (PR), stable disease (SD), and combinations thereof.

3. The method of claim 1, wherein the cancer is responsive to chemotherapy.

4. The method of claim 1, wherein the cancer has not progressed after chemotherapy.

5. The method of claim 1, wherein the cancer has not progressed after treatment with a PD-1 inhibitor or a PD-L1 inhibitor.

6. The method of claim 1, wherein the cancer has not progressed after chemotherapy and treatment with a PD-1 inhibitor or a PD-L1 inhibitor.

7. The method of claim 1, wherein the human has advanced or recurrent endometrial cancer.

8. The method of claim 1, wherein the human has TP53 wild-type endometrial cancer.

9. The method of claim 1, wherein the human has cyclin-dependent kinase inhibitor 2A (CDKN2A)-deleted or inactivated endometrial cancer.

10. The method of claim 1, wherein the human has endometrial cancer characterized by p16INK4a promoter hypermethylation and/or p14ARF promoter hypermethylation.

11. The method of claim 1, wherein the MDM2 inhibitor is a pharmaceutically acceptable salt of a compound of Formula (I).

12. The method of claim 1, wherein the compound of Formula (I) is administered once daily at a dose selected from the group consisting of about 15 mg, about 25 mg, about 30 mg, about 50 mg, about 60 mg, about 75 mg, about 90 mg, about 100 mg, about 120 mg, about 150 mg, about 175 mg, about 180 mg, about 200 mg, about 225 mg, about 240 mg, about 250 mg, about 275 mg, about 300 mg, about 325 mg, about 350 mg, about 360 mg, about 375 mg, and about 480 mg.

13. The method of claim 1, wherein the compound of Formula (I) is administered twice daily at a dose selected from the group consisting of about 15 mg, about 25 mg, about 30 mg, about 50 mg, about 60 mg, about 75 mg, about 90 mg, about 100 mg, about 120 mg, about 150 mg, about 175 mg, about 180 mg, about 200 mg, about 225 mg, about 240 mg, about 250 mg, about 275 mg, about 300 mg, about 325 mg, about 350 mg, about 360 mg, about 375 mg, and about 480 mg.

14. The method of any claim 1, wherein the human is treated with the compound of Formula (I) for a period selected from the group consisting of about 7 days, about 14 days, about 21 days, about 28 days, about 35 days, about 42 days, about 49 days, and about 56 days.

15. The method of claim 1, wherein the compound of Formula (I) is orally administered.

16. The method of claim 1, wherein the therapeutically effective amount of the MDM2 inhibitor is about 180 mg.

17. The method of claim 1, wherein the therapeutically effective amount of the MDM2 inhibitor is about 240 mg.

18. The method of claim 1, wherein the human is previously treated with platinum-based therapy and/or radiation therapy.

19. (canceled)

20. The method of claim 1, wherein the MDM2 inhibitor is administered intermittently.

21. The method of claim 20, wherein the MDM2 inhibitor is administered on days 1-7, followed by discontinuance of administration for days 8-28 on a 28-day cycle, optionally repeating the cycle of administration and discontinuation of administration for 2, 3, 4, 5, 6, 7, 8, 9, or 10 times.
